(19) United States
(12) Patent Application Publication
(10) Pub. No.: US 2022/0155438 A1
(43) Pub. Date: May 19, 2022

(54) AN ACOUSTIC COUPLING INTERFACE

(71) Applicant: IMEC VZW, Leuven (BE)

(72) Inventor: Xavier ROTTENBERG, Kessel-Lo (BE)

(21) Appl. No.: 17/438,694

(22) PCT Filed: Feb. 17, 2020
(86) PCT No.: PCT/EP2020/054016

§ 371 Date: Sep. 13, 2021

(30) Foreign Application Priority Data
Mar. 14, 2019 (EP) 19162705.8

(51) Int. Cl. G01S 15/89

(52) U.S. Cl. CPC G01S 15/8915; A61B 8/4494

(57) ABSTRACT

The present invention provides an acoustic coupling interface (1) for use between a flexible ultrasound transducing device (2) and a curved object (3) to be examined. The interface (1) is in the form of a sheet (4) having a bending flexibility that permits the sheet (4) to form a continuous contact with said curved object (3) during operation of the flexible ultrasound transducing device (2). Further, the sheet (4) comprises a bulk material (5) and a plurality of acoustic waveguiding structures (6) arranged in said bulk material (5), wherein the plurality of acoustic waveguiding structures (6) is for providing bidirectional coupling of ultrasound signals (7) emitted by the ultrasound transducing device (2).

### TECHNICAL FIELD

The present inventive concept relates to the field of ultrasonic examination. More particularly it relates to an acoustic coupling interface for use with a flexible ultrasound transducer.

### BACKGROUND

Large 2D arrays of ultrasound arrays have several applications in the medical market and for consumer electronics. Examples are medical imaging, gesture recognition, directed sound, fingerprint detection and mid-air haptics.

The standard structure of a piezoelectric micromachined ultrasound transducer (pMUT) is known in the art. A small drum is made, with a suspended membrane on top of a small cavity. The dimensions of this cavity in combination with the stiffness of the membrane will determine the resonance frequency of a particular MUT. As an example, the MUT may be driven by the piezo-electric effect (pMUT). By applying an AC electric field at the resonance frequency across a piezoelectric material, a stress difference between the piezo-electric material and the membrane is generated, and this will induce a vibration and the emission of an acoustical wave. Typical frequencies are in the range of 50 kHz to 20 MHz. Applications that use beam-forming to create a focal spot in emission or to image a small spot in receiving, require larger arrays of ultrasound transducers working together.

There is a need in the art for improved designs of ultrasound transducers and systems for examining curved object, e.g. for imaging or for obtaining data representative of features of an objects, since ultrasound transducers that are manufactured on flat rigid substrates may not be fit for scanning curved objects. The operator has to move and press the flat transducer against the curved object to be examined, which leads to difficulties in reproduction of images etc.

Flexible ultrasound transducer is known from e.g. US 20180168544, which discloses methods and systems for coupling a flexible transducer to an object. A transducer positioning device includes an inflatable bladder and a strap. The inflatable bladder may apply a force to a transducer array to maintain its position against the object when inflated. The strap may hold the bladder against the transducer array. Once in place, the bladder may be inflated with a fluid.

However, there is a need in the art for solutions that allow for improved ultrasonic examination of curved objects using flexible ultrasound transducers.

### SUMMARY

It is an object of the invention to at least partly overcome one or more limitations of the prior art. In particular, it is an object to provide a coupling interface for a flexible ultrasound transducing device that provides for improved ultrasonic examination of curved objects.

As a first aspect of the invention, there is provided an acoustic coupling interface for use between a flexible ultrasound transducing device and a curved object to be examined; wherein said interface is in the form of a sheet having a bending flexibility that permits the sheet to form a continuous contact with said curved object during operation of the flexible ultrasound transducing device, and wherein said sheet comprises a bulk material and a plurality of acoustic waveguiding structures arranged in said bulk material, wherein the plurality of acoustic waveguiding structures is for providing bidirectional coupling of ultrasound signals emitted by the ultrasound transducing device.

The acoustic coupling interface may be a disposable interface and is used between a flexible ultrasound transducing device and a curved object to be examined. The object being examined refers to the object in contact with the acoustic coupling interface during examination using a flexible ultrasound transducing device. This object may thus be curved, even though data representative of features that are not curved but resides within the examined curved object is gathered during examination.

The curved objects suitable may be objects comprising a curvature with a curvature radius of less than 20 cm, such as less than 10 cm. The curved object may be a part of a body of a patient, such as an arm or a leg. The acoustic coupling interface is a flexible interface that may follow the flexing of a flexible ultrasound transducer during ultrasonic examination of a curved object.

The acoustic coupling interface is further in the form of a sheet. The sheet of the acoustic interface generally consists of two planar surfaces, the first and second planar surface, oppositely arranged of each other, i.e. having normal vectors pointing in two different and parallel directions.

The first and second planar surfaces may thus extend in an X-Y plane and the sheet may a thickness defined in a Z-direction that is perpendicular to both X and Y directions. The sheet has further a bending flexibility, e.g. a bending flexibility in the X-Y plane in positive or negative Z-direction, that allows the acoustic coupling interface to bend around curved object and for a continuous contact with the curved object without breaking during examination of the curved object. An imaginary line drawn between two points on the surface of the sheet, said points being separated in the Z direction, is not straight but curved when the sheet is being flexed or bent in the Z-direction.

The bulk material of the acoustic interface comprises a plurality of acoustic waveguiding structures. The sheet may thus consist of the bulk material.

The bulk material may itself be a flexible material.

The bulk material may comprise or consist of a rubber or a polymeric material. Thus, in embodiments of the first aspect, the bulk material comprises a polymer. The bulk material may be a rubber or comprise a rubber. Moreover, the bulk material may be selected from the group consisting of SU-8, silicon nitride and polyimide.

Further, the bulk material may be a layered material. Thus, the bulk material may comprise a multi-layered structure comprising individual layers stacked on top of each other. One such individual layer does not need to be flexible, but the whole multi-layered structure may be flexible. As an example, individual layers of the multi-layered structure may comprise or consist of silicon oxide.

The waveguiding structures may comprise acoustic waveguides, such as an array of acoustic waveguides. Furthermore, the acoustic waveguiding structures may comprise a set of acoustic scatterers arranged in the bulk material that together work or function as an acoustic waveguiding structure.

The plurality of acoustic waveguiding structures is for providing bidirectional coupling of ultrasound signals emitted by the ultrasound transducing device. Thus, the acoustic coupling interface facilitates transmittance of ultrasound signals emitted by a flexible ultrasound transducer into the object being examined and allows for the resultant echo signals from the object being transmitted back to the flexible ultrasound transducer. The acoustic waveguiding structures may therefore function as an acoustic lens for the ultrasonic waves emitted by an ultrasonic transducer and for the echo signal received from the object being examined.

The first aspect of the invention is based on the insight that the flexible acoustic interface permits good acoustic contact between the object that is examined, such as a person, and a flexible ultrasound transducer, such as a flexible ultrasound transducer comprising an array of ultrasonic transducing elements. The flexible acoustic interface thus transfers the shape or curvature of the object being examined to the flexible ultrasound transducer. The flexible acoustic interface may further function as an alternative or complement to a gel that is conventionally used when performing ultrasonic examination of e.g. a human body. Due to the waveguiding structures, the acoustic interface couples acoustic waves in a way that allows non-uniform, and possibly non-sticking, contact with the object being examined.

Further, the acoustic interface may function as a disposable patch, which permits reuse of the flexible ultrasound transducer.

In embodiments of the first aspect, the sheet has a first planar surface arranged for contacting said flexible ultrasound transducing device and a second planar surface, opposite said first surface, arranged for contacting said curved object to be examined, and wherein said sheet has a bending flexibility such that the surface profiles of both the first and second planar surfaces are altered with the second planar surface conforming to the curved object when the sheet is in contact with said curved object during operation.

The flexibility and thickness of the sheet may such that the surface profiles of the first and second planar surfaces are affected during examination, i.e. when a flexible transducer is pressed against the object being examined. Consequently, this aids in “transferring” the curvature of the object to a flexible ultrasound transducer.

As an example, the first and second planar surfaces may have a length and width that both are at least five times the thickness of the sheet, such as at least 10 times, such as at least 20 times, such as at least 50 times, the thickness of the sheet. Thus, both the length of the sheet as well as the width of the sheet may be at least five times longer than the thickness.

As an example, the sheet may have a length and width that is at least 0.2 cm, such as at least 1 cm, such as at least 5 cm, such as at least 10 cm, such as at least 50 cm.

As an example, the sheet may have a surface area that is between 0.20 cm² and 0.50 cm². As a further example, the sheet may have a surface area that is between 1 cm² and 25 cm². As a further example, the sheet may have a surface area that is between 30 cm² and 70 cm².

As a further example, the sheet may have a surface area that is at least 80 cm², such as about 100 cm².

Moreover, the sheet may have a surface area that is more than 0.10 m², such as more than 0.20 m².

Further, the sheet may have a thickness that is between 10 μm and 1 cm, such as between 0.5 mm and 1.0 mm.

As an example, the first and second planar surfaces may extend in an X and Y direction and wherein the sheet has a thickness extending in a Z direction that is perpendicular to the X and Y directions. The sheet may have a flexibility such that the sheet may be bent in the Z direction with a bend angle that is at least 30 degrees, such as at least 45 degrees, such as at least 90 degrees.

In embodiments of the first aspect, the sheet has a bending flexibility that permits the sheet to be bent with a radius of curvature (Rc) that is less than 5 cm, preferably less than 3 cm, more preferably less than 2 cm.

In other embodiments of the first aspect, the sheet has a bending flexibility that permits the sheet to be bent with a radius of curvature (Rc) that is between 10 and 30 cm, such as between 15 and 20 cm. The radius of curvature may be defined when bending in positive or negative Z-direction, i.e. the direction along the normal to the first and/or second planar surfaces. The minimum radius of curvature may be less than 5 cm, such as 1-4 cm.

Furthermore, in embodiments of the first aspect, the whole sheet or the bulk material has a flexural modulus (modulus of elasticity) that permits the sheet to form a continuous contact with said curved object during operation of the flexible ultrasound transducing device.

The flexural modulus of a material is a physical property denoting the ability for that material to bend. In mechanical terms, it is the ratio of stress to strain during a flexural deformation, or bending. If the sheet is made of plastics, the type of polymer, molecular weight and thickness may affect the flexibility.

As an example, the whole sheet or the bulk material may have a flexural modulus that is less than 50 GPa, such as less than 20 GPa, thereby permitting the sheet to form a continuous contact with said curved object during operation of the flexible ultrasound transducing device.

The flexural modulus of a material may be measured using a known 3-point analysis on a rectangular beam of the material having width w and height h. Parameter L defines a length between two support points on a first side of the beam and a force F is applied on the other side of the beam. The displacement or deflection d is measured and the flexural modulus, E_{bend} (force per area) is calculated using

Ebend=(L³F)/(4 wh³d)

In embodiments of the first aspect, the plurality of acoustic waveguiding structures are arranged in an array in the bulk material. The array may be one-dimensional, two-dimensional and/or three dimensional.

As an example, the plurality of acoustic waveguiding structures may comprise more than 20, such as more than 50, individual acoustic waveguiding structures or acoustic waveguides.

In embodiments of the first aspect, the sheet is a stretchable sheet. Thus, the sheet of the acoustic coupling interface may be of a material that can withstand strain reversibly. Thus, the sheet may also comprise or consist of an elastic material.

However, the sheet may also comprise or consist of a non-elastic material.

In embodiments of the first aspect, the plurality of acoustic waveguiding structures comprises waveguides having an elongated form.

The waveguides may thus be in the form of pillar extending through part or the whole bulk material.

In embodiments of the first aspect, the plurality of acoustic waveguiding structures comprises an arrangement of alternating bulk material and another material different from the bulk material, said arrangement extending from the first planar surface to the second planar surface.

As an alternative, the acoustic waveguiding structures may be entirely enclosed by the bulk material.

The material different from the bulk material may be voids or a material having a different acoustic impedance than the bulk material

The arrangement of alternating bulk material and said “another material” may form a three-dimensional array of discrete elements of said “another material” within the bulk material. The discrete elements, such as a row of discreet elements in the three-dimensional array, may function together as an acoustic waveguiding structure.

As an example, the plurality of acoustic waveguiding structures may comprises waveguides extending from the first planar surface to the second planar surface. Consequently, the acoustic waveguiding structure may extend in the whole Z-direction or through the whole thickness of the sheet. The waveguides may thus form a plurality of pillars extending within the bulk material though the thickness of the sheet in a direction substantially perpendicular to the first or second planar surface of the sheet.

In embodiments of the first aspect, the plurality of acoustic waveguiding structures comprises a solid material different than the bulk material. The solid material different than the bulk material may have a different acoustic impedance than the bulk material. The solid material may be a metallic material or a polymeric material.

As an example, the solid material of the plurality of acoustic waveguiding structures may protrudes out from the first and/or second planar surface. Thus, the plurality of acoustic waveguiding structures may only protrudes out from the first planar surface of the sheet, they may protrude from both the first and second planar surface of the sheet or they may protrude only out from the second surface of the sheet. This may be advantageous in that the protruding waveguiding structures may facilitate adhesion to another surface during operation, such as to the surface of the curved object being examined or to the surface of a flexible ultrasound transducer.

The protruding waveguiding structures of a solid material may thus form an array of protruding elements that increases the adhesive properties of the acoustic coupling interface.

In embodiments of the first aspect, the plurality of acoustic waveguiding structure structures comprises internal walls in the bulk material so as to define waveguiding structures in the form of voids in the bulk material. As discussed above, the voids may have an elongated form and extend from the first to the second planar surface of the sheet.

As a second aspect of the invention, there is provided, a system for creating data representative of features of a curved object comprising
a flexible ultrasound transducing device, and
an acoustic coupling interface according to the first aspect above configured to be removably attached to said ultrasound transducing device such that ultrasound signals emitted by the transducing device are
transmitted into said object and resultant echo signals from the object are
transmitted back to the ultrasound transducing device.

This aspect may generally present the same or corresponding advantages as the former aspect.

The data representative of features of a curved object that is generated may be used for imaging of the curved object.

The flexible ultrasound transducer may comprise an array of ultrasound transducing elements. The ultrasound transducing elements may be configured for generating ultrasonic energy propagating along a main transducer axis (Z). The flexible ultrasound transducing device may comprise a first outer surface facing said curved object during examination and having a normal vector that is parallel to the main transducer axis. The acoustic coupling interface may thus be configured to be removably attached to such a first outer surface of the flexible ultrasound transducer.

The first outer surface of the ultrasound transducer may have a surface area that is at least 100 cm², such as at least 400 cm². Thus, the array of transducing elements of such a flexible ultrasound transducer may cover an area that is at least 100 cm², such as at least 400 cm².

The system may thus be provided as a kit with a flexible ultrasound transducer and at least one acoustic coupling interface according to the first aspect above. The flexible ultrasound transducer may be reused whereas the acoustic coupling interface may be a disposable interface that is changed between ultrasonic examinations. The system is advantageous e.g. in that provides for ultrasonic examination, such as imaging, without having to use a traditional gel for acoustic coupling between the object being examined and the ultrasound transducer. As a third aspect of the invention, there is provided a method of obtaining data representative of features of an object comprising
subjecting the object to ultrasound signals using a system according to the second aspect above; and
analysing the resultant echo signals from the object and thereby obtaining data representative of features of said object based on the resultant echo signals.

This aspect may generally present the same or corresponding advantages as the former aspects discussed above. The object may be a curved object, such as a curved object comprising a curvature having a curvature with a curvature radius of less than 20 cm, such as less than 10 cm. The curved object may be a part of a body of a patient, such as an arm or a leg

The step of analysing the resultant echo signals may comprise forming an image of a part of the object being examined, such as the inside of the object being examined.

In embodiments of the third aspect, the step of subjecting the object to ultrasound signals is performed with a direct contact of the acoustic coupling interface and the object and/or a direct contact of the acoustic coupling interface and the flexible ultrasound transducing device.

As an example, the step of subjecting the object to ultrasound signals may be performed with a direct contact of the acoustic coupling interface and the object, e.g. without any gel between the object and the acoustic coupling interface.

As a further example, the step of subjecting the object to ultrasound signals may performed with a direct contact of the acoustic coupling interface and the flexible ultrasound transducing device, e.g. without any gel between the acoustic coupling interface and the flexible ultrasound transducing device.

As another example, the step of subjecting the object to ultrasound signals may performed with a direct contact of the acoustic coupling interface and the flexible ultrasound transducing device and a direct contact of the acoustic coupling interface and the object, e.g. without any gel between the acoustic coupling interface and the flexible ultrasound transducing device and without any gel between the object and the acoustic coupling interface.

However, the step of subjecting the object to ultrasound signals may as an alternative be performed with an indirect contact of the acoustic coupling interface and the object and/or an indirect contact of the acoustic coupling interface and the flexible ultrasound transducing device. Thus, a gel may be used between the acoustic coupling interface and the object or between the acoustic coupling interface and the flexible ultrasound transducing device. As a further example, a gel may be used between the acoustic coupling interface and the object as well as between the acoustic coupling interface and the flexible ultrasound transducing device.

### BRIEF DESCRIPTION OF THE DRAWINGS

The above, as well as additional objects, features and advantages of the present inventive concept, will be better understood through the following illustrative and non-limiting detailed description, with reference to the appended drawings. In the drawings like reference numerals will be used for like elements unless stated otherwise.

FIG. 1 is a perspective view of a schematic illustration of an acoustic coupling interface of the present disclosure.

FIGS. 2a-f are illustrative embodiments of the acoustic waveguiding structures arranged in the bulk material of the sheet.

FIG. 3 is a schematic illustration of the bend angle and the radius of curvature when the acoustic coupling interface is bent in negative Z-direction.

FIGS. 4a-d shows illustrative embodiments of a system s for creating data representative of features of a curved object during examination of an object.

FIG. 5 is a schematic illustration of a method of obtaining data representative of features of an object.

### DETAILED DESCRIPTION

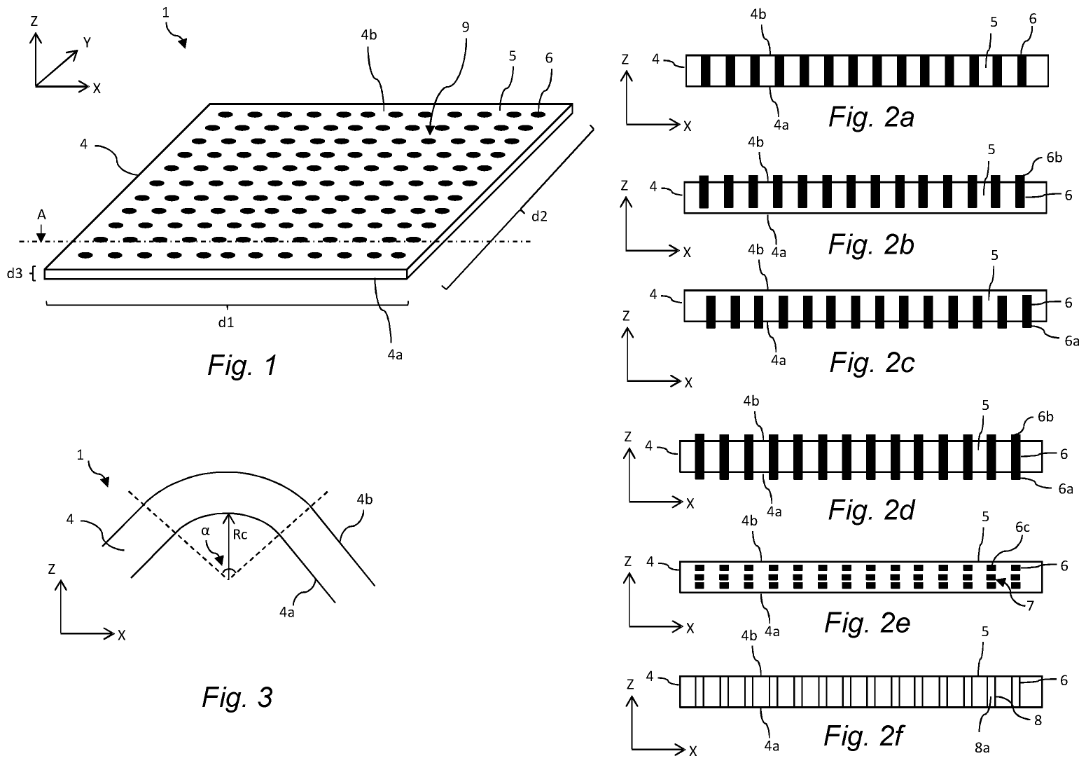

FIG. 1 shows a schematic example of an acoustic coupling interface 1 according to the present disclosure. The interface 1 is in the form of a sheet 4 that extends in an X-Y plane, with a thickness extending in a Z-direction perpendicular to both X and Y directions. The sheet 4 has a first planar surface 4a and a second planar surface 4b opposite the first planar surface 4a, and since the interface 1 is for use between a flexible ultrasound transducing device 2 and an object, such as a curved object 3, one of the planer surfaces may during use face the object 3 that is examined whereas the opposite planar surface faces the ultrasound transducing device. Consequently, the sheet 4 has a first planar surface 4a arranged for contacting said flexible ultrasound transducing device 2 and a second planar surface 4b, opposite said first surface 4a, arranged for contacting said curved object 3 to be examined.

The sheet 4 has in this example a rectangular or quadratic shape with a length d1 in the X direction of about 5-20 cm, such as about 10 cm and a length d2 in the Y-direction of about 5-20 cm, such as about 10 cm. The sheet is further thin in relation to the surface areas of the first 2a and second 2b planar surfaces, such as having a thickness d3 in the Z-direction of about 0.1 mm-1.0 mm. Thus, the first 4a and second 4b planar surfaces may have a length and width that both are at least fifty times the thickness of the sheet.

Moreover, the sheet 4 comprises a bulk material 5 and a plurality of acoustic waveguiding structures 6 arranged in the bulk material (5). In the example of FIG. 1, the acoustic waveguiding structures 6 are arranged as a two-dimensional array 9 in the bulk material 5.

The plurality of acoustic waveguiding structures 6 is for providing bidirectional coupling of ultrasound signals 7 emitted an ultrasound transducing device 2 during examination of an object 3.

FIGS. 2a-f shows different embodiments of acoustic waveguiding structures 6.

FIGS. 2a-f are section view of a sheet 4, e.g. a section view along line A of the sheet in FIG. 1.

FIG. 2a shows a schematic embodiment of acoustic waveguiding structures 6 arranged within the bulk material 5. The waveguiding structures 6 have an elongated form extending from the first planar surface 4a to the second planar surface 4b. Thus, the waveguiding structures 6 extend in the Z direction, i.e. in the direction in which the thickness of the sheet 4 is defined. The elongated waveguiding structures, or elongated waveguides 6, may have any suitable form, such as in the form of cylinders or having convex or concave outer forms.

FIG. 2b shows a schematic embodiment of acoustic waveguiding structures 6 arranged within the bulk material 5. In this example, the waveguiding structures 6 protrude from the second planar surface 4b.Thus, the waveguiding structures 6 comprises a portion 6a that extends or protrudes out from the second planar surface 6b. The second planar surface 4b may thus comprise an array of protruding portions 6b. The protruding portions 6a may aid in keeping the contact between the acoustic coupling interface 1 with an object 3 during examination by making the second outer planar surface 4b of the sheet 4 more sticky, i.e. by increasing the friction between the interface 1 and the object 3 during examination.

The acoustic waveguiding structures 6 may also protrude from the first planar surface 4a. This example is shown in FIG. 2c, in which the waveguiding structures 6 comprises a portion 6a that extends or protrudes out from the first planar surface 6a. The first planar surface 4a may thus comprise an array of protruding portions 6a. The protruding portions 6a may aid in keeping the contact between the acoustic coupling interface 1 and a flexible ultrasound transducing device 2 during examination by making the first outer planar surface 4a of the sheet 4 more sticky, i.e. by increasing the friction between the interface 1 and the flexible ultrasound transducing device 2 during examination.

The acoustic waveguiding structures 6 may also protrude both from the first planar surface 4a and the second planar surface 4b. Such an example is shown in FIG. 2d, in which the acoustic waveguiding structures 6 both comprises a portion 6a protruding out from the first planar surface 6a and a portion 6a protruding out from the second planar surface 6b.

Having such an arrangement of protruding portions 6a and/or 6b, using a gel between interface 1 and transducer array 2, or a gel between interface and the object 3 that is examined, may be unnecessary.

FIG. 2e shows a schematic embodiment of the sheet 4 in which plurality of acoustic waveguiding structures 6 is arranged within the bulk material 6 and comprises an arrangement 7 of alternating bulk material 6 and another material 6c different from the bulk material 6. The arrangement 7 extends in the Z-direction from the first planar surface 4a to the second planar surface 4b. The material 6c other than the bulk material is thus arranged as discrete elements within the bulk 4, e.g. along imaginary straight lines extending from the first planar surface 4a to the second planar surface 4b.The size of the discrete elements and the distance between adjacent discrete elements makes the arrangement 7 work as a guiding structure for ultrasonic waves propagating through the sheet 4.

FIG. 2f shows a further schematic embodiment of a sheet 4 comprising a plurality of acoustic waveguiding structures similar to the embodiment discussed in relation to FIG. 2a above, but the acoustic waveguiding structures 6 are in the form of voids 8a arranged within the bulk material 5. Thus, the plurality of acoustic waveguiding structures 6 comprises in this example internal walls 8 in the bulk material 6 so as to define waveguiding structures in the form of voids 8a in the bulk material 6.

The acoustic waveguiding structures 6 may be of a material having a different acoustic impedance than the bulk material 6. Thus, the plurality of acoustic waveguiding structures 6 may comprise a solid material different than the bulk material 5.

The acoustic waveguiding structures 6 may be or comprise a metal or polymer, and may form three-dimensional acoustic impedance objects within the bulk material 6.

The bulk material 6 may comprise a polymer, such as polyimide (PI). The bulk material may be a flexible material so that the sheet 4 becomes flexible. Thus, the sheet 4 may be flexible so as to form a continuous contact with a curved object 3 during operation of a flexible ultrasound transducing device 2. As an example, the sheet 4 may have a bending flexibility such that the surface profiles of both the first 4a and second b planar surfaces are altered during examination. The second planar surface, which is the surface in contact with or closest to the object being examined, may conform to the curved object 3 when the sheet 4 is in contact with a curved object 3 during examination. However, the sheet 4 may be flexible enough so that also the first planar surface 4a may conform to the curved object during examination.

Consequently, the acoustic coupling interface 1 may facilitate transfer of the surface profile of the object 3 being examined to a flexible ultrasound transducing device during examination.

FIG. 3 illustrates how the flexibility of the sheet 4 may be measured. In analogy with what is shown in FIG. 1, the first 4a and second 4b planar surfaces extend in an

X and Y direction and the sheet 4 has a thickness extending in a Z direction that is perpendicular to the X and Y directions. The sheet may have a flexibility and such that it may be bent in positive or negative Z direction with a bend angle (a) that is at least 30 degrees without breaking. The thickness of the sheet 4 may in combination with the material of the bulk material, be the most important factor influencing the flexibility of the sheet 4. When bending the sheet 4, the “breaking” may refer to cracks appearing on the outer surface during bending, i.e. the surface having an area under tension during the bending. In the Example shown in FIG. 3, this area would be the surface area of the second planar surface 2b.

Further, the radius of curvature Rc may be less than 5 cm without the sheet 4 breaking. The radius of curvature may be the inside curvature during bending. Thus, in FIG. 3, the radius of curvature is measured on the first planar surface 3a since the sheet 4 is bent in negative Z-direction.

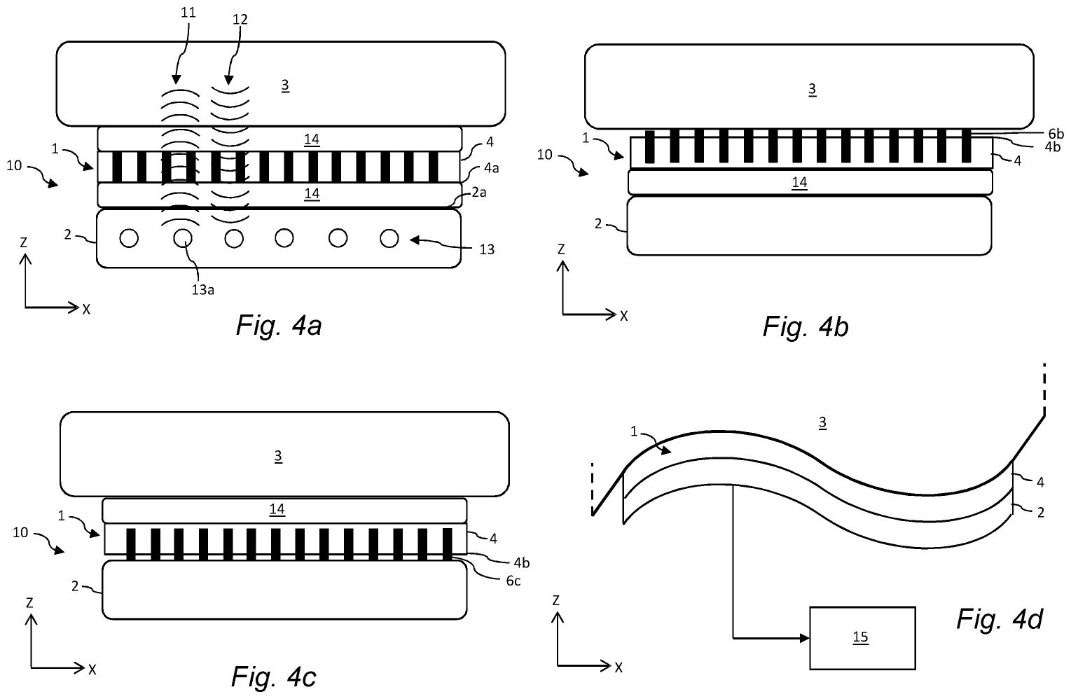

FIGS. 4a-4d show different schematic and illustrative embodiments of a system 10 for creating data representative of features of an object 3.

As shown in FIG. 4a, the system 10 comprises a flexible ultrasound transducing device 2. This device 2 comprises an array 13 of individual ultrasound transducing elements 13a. The ultrasound transducing device 2 is for both emitting ultrasonic waves 11 and for receiving echo signals 12 from the object 3 being examined. The individual ultrasound transducing elements 13a of the array 13 may be micromachined ultrasound transducers (MUT), which are known in the art. Such element 13a may be formed by processing a small drum with a suspended membrane on top of a small cavity. The dimensions of this cavity in combination with the stiffness of the membrane will determine the resonance frequency of a particular

MUT. The MUT may be driven by the piezo-electric effect, forming a pMUT, which functions by applying an AC electric field at the resonance frequency across a piezoelectric material to generate a stress difference between the piezo-electric material and the membrane. This will induce a vibration and the emission of an acoustical wave. Typical frequencies are in the range of 50 kHz to 20 MHz. This translates into wavelengths ranging from 1 cm down to <100 um. Applications that use beam-forming to create a focal spot in emission or to image a small spot in receiving, may require larger arrays of ultrasound transducing elements 13a working together.

The system 10 further comprises an acoustic coupling interface 1 as disclosed herein above. The interface 10 is removably attached onto an outer surface 2a of the transducer 2 between the transducer 2 and the object 3 being examined. The object 13 may be a part of a body, such as an arm or a leg. The acoustic coupling interface 1 thus provides for bidirectional coupling such that the ultrasound signals 11 emitted by the transducer 2 are transmitted into the object 3 and the resultant echo signals 12 from the object 3 are transmitted back to the array 13a of the transducer 2.

The ultrasound transducing elements 13 a are configured for generating ultrasonic energy propagating along a main transducer axis parallel to Z-axis, and the flexible ultrasound transducing device 2 may comprise a first outer surface 2a facing the curved object 3 during examination. This first outer surface 2a of the transducer 2 thus has a normal vector that is parallel to the main transducer axis, and the acoustic coupling interface 1 is arranged between the object 3 and the transducer 2 with the first outer planar surface 4a of the sheet 4 facing the first outer surface 2a of the transducer 2. In the embodiments illustrated in FIG. 2a, a gel 14 is applied between the flexible ultrasound transducer 2 and the acoustic coupling interface 1 and between the acoustic coupling interface 1 and the object 14 being examined.

FIG. 4b shows an embodiment of the system 10 in which the waveguiding structures 6 of the acoustic coupling interface 1 has protruding portions 6a on the second planar surface 4b of the sheet 4. This provides for ultrasonic examination of the curved object 3 without having a gel between the acoustic coupling interface and the object 3. The protrusions 6a may facilitate adhesion of the interface 1 and the whole system 10 to the object 3 being examined. It may also be beneficial is certain applications to have a dry contact between object 3 and the system 10.

FIG. 4c shows an embodiment of the system 10 in which the waveguiding structures 6 of the acoustic coupling interface 1 has protruding portions 6a on the first planar surface 4a of the sheet 4. This provides for ultrasonic examination of the curved object 3 without having a gel between the acoustic coupling interface and the ultrasonic transducer. In analogy with the embodiment shown in FIG. 4b, the protrusions 6a may aid in the adhesion of the interface 1 to the flexible ultrasound transducer 2. It may be beneficial is certain applications to have a dry contact between the ultrasound transducer 2 and a disposable acoustic coupling interface 1.

FIG. 4d shows the use of the system 10 for examining a curved object 3. As illustrated in FIG. 4d, the flexibility of the sheet 4 makes it possible for the whole interface 1 to conform to the curved surface of the object 3 during examination, Further, the flexibility of the acoustic coupling interface 1 also makes it possible for the flexible ultrasound transducer 2 to conform to the curvature of the curved object 2 during examination. In this embodiment, neither a gel between the acoustic coupling interface 1 and the ultrasonic transducer 2 nor a gel between the acoustic coupling interface and the object 3 is used. Consequently, the acoustic coupling interface provides for a dry contact during examination and thus the exclusion of a gel, which may be practical benefit in various applications.

The system as disclosed in FIGS. 4a-4c is for creating data representative of features of an object 3. The data obtained may for example be used by a control unit 15 in the system 10 for creating an image of the internal of the object 3. The control unit 15 may comprise a communication interface, such as a transmitter/receiver, via which it may receive and transmit data from and to the ultrasound transducer 2. The control unit 15 may comprise a processing unit, such as a central processing unit, for calculating image parameters using the data obtained from the ultrasound transducer 2. Such a processing unit may be configured to execute computer code instructions which for instance may be stored on a memory.

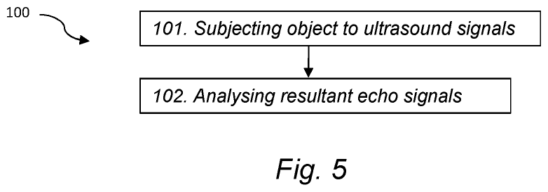

FIG. 5 schematically illustrates a method 100 of obtaining data representative of features of an object. The method 100 comprises subjecting 101 the object to ultrasound signals using a system 10 as disclosed herein above for creating data representative of features of an object 3.

Further, the method 100 comprises analysing (102) the resultant echo signals from the object 3 and thereby obtaining data representative of features of said object based on the resultant echo signals. The analyses may for example be performed by a control unit as discussed in relation to FIG. 4d above.

The step 101 of subjecting the object to ultrasound signals may be performed with a direct contact of the acoustic coupling interface 1 and the object 3, such as shown in FIG. 4b, with a direct contact of the acoustic coupling interface 1 and the flexible ultrasound transducing device 2, such as shown in FIG. 4c above, or with a direct contact of the acoustic coupling interface 1 with the object 3 and the ultrasound transducer 2, such as shown in FIG. 4d.

In the above the inventive concept has mainly been described with reference to a limited number of examples. However, as is readily appreciated by a person skilled in the art, other examples than the ones disclosed above are equally possible within the scope of the inventive concept, as defined by the appended claims.

1. An acoustic coupling interface for use between a flexible ultrasound transducing device and a curved object to be examined, wherein said interface is in the form of a sheet having a bending flexibility that permits the sheet to form a continuous contact with said curved object during operation of the flexible ultrasound transducing device, and wherein said sheet comprises
a bulk material and a plurality of acoustic waveguiding structures arranged in said bulk material, wherein the plurality of acoustic waveguiding structures is for providing bidirectional coupling of ultrasound signals emitted by the ultrasound transducing device.

2. An acoustic coupling interface according to claim 1, wherein said sheet has a first planar surface arranged for contacting said flexible ultrasound transducing device and a second planar surface, opposite said first surface, arranged for contacting said curved object to be examined, and wherein said sheet has a bending flexibility such that the surface profiles of both the first and second planar surfaces are altered with the second planar surface conforming to the curved object when the sheet is in contact with said curved object during operation.

3. An acoustic coupling interface according to claim 2, wherein the first and second planar surfaces have a length and width that both are at least five times the thickness of the sheet.

4. An acoustic coupling interface according to claim 1, wherein the sheet has a bending flexibility that permits the sheet to be bent with a radius of curvature (Rc) that is less than 5 cm.

5. An acoustic coupling interface according to claim 1, wherein the plurality of acoustic waveguiding structures comprises waveguiding structures having an elongated form.

6. An acoustic coupling interface according to claim 2, wherein the plurality of acoustic waveguiding structures comprises an arrangement of alternating bulk material and another material different from the bulk material, said arrangement extending from the first planar surface to the second planar surface.

7. An acoustic coupling interface according to claim 2, wherein the plurality of acoustic waveguiding structures comprises waveguiding structures extending from the first planar surface to the second planar surface.

8. An acoustic coupling interface according to claim 1, wherein the plurality of acoustic waveguiding structures comprises a solid material different than the bulk material.

9. An acoustic coupling interface according to claim 7, wherein the plurality of acoustic waveguiding structures protrudes out from the first and/or second planar surface.

10. An acoustic coupling interface according to claim 1, wherein the plurality of acoustic waveguiding structures comprises internal walls in the bulk material so as to define waveguiding structures in the form of voids in the bulk material.

11. An acoustic coupling interface according to claim 1, wherein the bulk material comprises a polymer.

12. A system for creating data representative of features of a curved object comprising:
a flexible ultrasound transducing device, and
an acoustic coupling interface according to claim 1 configured to be removably attached to said ultrasound transducing device such that ultrasound signals emitted by the transducing device are transmitted into said object and resultant echo signals from the object are transmitted back to the ultrasound transducing device.

13. A system according to claim 12, wherein said flexible ultrasound transducing device comprises an array of ultrasound transducers.

14. A method of obtaining data representative of features of a curved object comprising:
subjecting the object to ultrasound signals using a system according to claim 12;
analysing the resultant echo signals from the object and thereby obtaining data representative of features of said object based on the resultant echo signals.

15. A method according to claim 14, wherein the step of subjecting the object to ultrasound signals is performed with a direct contact of the acoustic coupling interface and the object and/or a direct contact of the acoustic coupling interface and the flexible ultrasound transducing device.
